(19) United States
(12) Patent Application Publication
(10) Pub. No.: US 2015/0245986 A1
(43) Pub. Date: Sep. 3, 2015

(54) Foam improvement of soap containing compositions

(71) Applicant: RHODIA OPERATIONS, Paris (FR)

(72) Inventors: Nikolay Christov, Singapore (SG); Tobias Futterer, Singapore (SG)

(73) Assignee: RHODIA OPERATIONS, Paris (FR)

(21) Appl. No.: 14/429,493

(22) PCT Filed: Sep. 26, 2013
(86) PCT No.: PCT/EP2013/070029

§ 371 Date: Mar. 19, 2015

(30) Foreign Application Priority Data
Oct. 1, 2012 (IB) PCT/IB2012/001919

(51) Int. Cl. A61K 8/41; A61K 8/36; A61K 8/73; A61Q 5/02; A61Q 19/10

(52) U.S. Cl. CPC A61K 8/416; A61Q 5/02; A61Q 19/10; A61K 8/731; A61K 8/737; A61K 8/361; A61K 2800/5428; A61K 2800/5426; A61K 2800/596

(57) ABSTRACT

The present invention concerns the use of a combination of an amphoteric surfactant and a cationic polymer derivative of polysaccharide to increase the foam volume and foam quality of soap containing compositions. Said advantage may be used to produce cleansing products like cleansing liquid, paste, gel or foam, body shampoo or hair shampoo.

The present invention concerns the use of a combination of an amphoteric surfactant and a cationic polymer derivative of polysaccharide to increase the foam volume and foam quality of soap containing compositions. Said advantage may be used to produce cleansing products like cleansing liquid, paste, gel or foam, body shampoo or hair shampoo.

### BACKGROUND OF THE INVENTION

Soaps representing salts of fatty acids with alkali are a mainstay for many skin cleansers. When properly formulated they provide rich and creamy lather together with excellent rinsability and squeaky clean perception. However soap-based compositions are relatively harsh to the skin with tight skin feel, have problems with stability in liquid form and do not foam well in hard water and form lime soap upon rinsing.

In order to address these issues various surfactants, polymers and solvents have been added to the soaps since decades. Although some of the major problematic issues could be resolved in this way, the composition cost is usually increased due to the additional ingredients. Also very often the improvement of one property impairs another one. For example the addition of high concentration of surfactants may increase the product mildness and the foaminess, especially in hard water, but could deteriorate the foam creaminess and the product rinsability. Similarly the addition of high amount of polymers, especially cationic ones, may increase the foam creaminess and the product mildness and stability, but will affect also the product rinsability and the wet skin afterfeel.

Creating a cost effective, mild and stable soap-based composition with good foamability and rinsability and with pronounced foam creaminess is still a big challenge to the formulators.

### INVENTION

Unexpectedly, it appears now that the combination of an amphoteric surfactant of formula (I) with a cationic polymer derivative of polysaccharide has a strong synergistic action in soap-based formulations, imparting mildness to the composition, creating rich and creamy foam upon application with excellent skin conditioning properties and leaving the skin with moist feel.

The present invention concerns then the use of a combination of a compound of formula (I) and a cationic polymer derivative of polysaccharide to increase the foam volume and foam quality of a soap containing aqueous composition; said composition comprising at least:

(1) 0.5 to 85% by weight, preferably 5 to 35% by weight, of carboxylic acids having 8 to 24 carbon atoms, and salts thereof;
(2) 0.5 to 15% by weight of an amphoteric surfactant of formula (I) as follows:

R¹—(CONH)ₐ—(CH₂)_{b}—N⁺(R²)(R³)(R⁴)Z⁺ (I)

wherein:
a is 0 or 1;
b is comprised between 1 and 3;
R¹ is an alkyl or alkenyl hydrocarbon chain comprising from 7 to 21 carbon atoms;
R² is an alkyl group having 1 to 3 carbon atoms or —(CH₂)_{c}OH with c is comprised between 1 and 3;
R³ is H, an alkyl group having 1 to 3 carbon atoms, or —(CH₂)_{d}COO⁻ with d is comprised between 1 and 3;
R⁴ is —(CH₂)ₑCOO⁻ with e is comprised between 1 and 3; or —(CH₂)_{f}CH(OH)—CH₂—SO₃⁻ with f is comprised between 1 and 3; and
Z is a monovalent cation.
   (3) 0.01 to 5% by weight, preferably 0.02 to 2% by weight, of a cationic polymer derivative of polysaccharides, preferably cationic guars;
   and each components of the composition are expressed in percent by weight in relation with the total weight of the composition.

The present invention is then relative to the use of a compound of formula (I) and a cationic polymer derivative of polysaccharide to increase the foam volume and foam quality of a soap containing aqueous composition. The invention is also relative to the use of a combination of components (1), (2) and (3) to obtain a foam product.

The present invention also concern a method to produce foaming product by using a composition comprising at least the above identified components (1), (2) and (3).

To increase the foam quality for the purposes of this invention is considered to provide a longer residence time when the foam goes through a sieve. This well-known test, notably as described in the experimental part, provides information about the foam viscosity and foam elasticity, as well for the foam-surface friction, which are among the main factors affecting the consumer perception about the foam quality.

Compositions of the invention may be formulated for washing skin and/or hair, for example, bath or shower gels, handwashing compositions, facial washing compositions, pre- and post-shaving products, and rinse-off and wipe-off skin care products, and mainly to produce cleansing foam, body shampoo and hair shampoo.

### DETAILS OF THE INVENTION

### Component (1)

The carboxylic acids have 8 to 24, preferably 12 to 18 carbon atoms. Preferred components (1) are the alkali metal or alkanol ammonium salts of aliphatic alkane- or alkene monocarboxilic acids. The suitable cations may be alkaline metals such as sodium and potassium, basic amino acids, organic ammonium compounds such as mono-, di- and tri-ethanol ammonium and the like.

Soaps may be made by saponification of natural fats and oil or by complete or partial neutralization of fatty acids and mixtures thereof. Alternatively, the soaps could be introduced directly into the compositions rather than being prepared in situ. The neutralization degree of the carboxylic acids is preferably 60 to 100%, more preferably 65 to 95%.

Generally, when carboxylic acids of the invention are neutralized using, for example, alkali metal hydroxide or carbonate, fatty acid soaps are made. Examples of compounds which may be used to neutralize are alkali metal hydroxides or carbonates.

Examples of carboxylic acids of the invention which may be used include lauric acid (C12), myristic acid (C14), palmitic acid (C16) and stearic acid (C18) or mixture thereof. Different grades of fatty acid mixtures produced by splitting and distillation of oils and fats could be also used in various combinations.

When the soap is made by in situ saponification than the coconut and palm kernel oils and the tallow are preferred. A combination of lauric acid (C12), myristic acid (C14) and palmitic acid (C16) is highly preferred according to the present invention.

### Component (2)

a is 0 or 1 and preferably 1. b may be comprised between 1 and 3 and is generally equal to 2 or 3. c is comprised between 1 and 3 and is generally equal to 2. d and e are independently comprised between 1 and 3 and are generally equal to 1. f is comprised between 1 and 3 and is generally equal to 1.

R¹ is an alkyl or alkenyl hydrocarbon chain comprising from 7 to 21 carbon atoms, preferably comprising from 11 to 17 carbon atoms, and may be derived from coconut, palm or a coconut/palm blend.

“Alkyl” as used herein means a straight or branched chain saturated aliphatic hydrocarbon.

“Alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbon atoms of the alkenyl group.

The suitable monovalent cation Z may be as example an alkaline metal such as sodium and potassium, or mono- di- or tri-ethanolammonium

According to the present invention, compounds of formula (I) may be alkylamphocarboxylates, alkylamphosulfonates, alkyl or alkylamidopropyl hydroxysultaine and alkyl or alkylamidopropyl betaines.

In a first embodiment, preferred compounds of formula (I) are alkylamphocarboxylates, that may be chosen in the group consisting of: sodium lauroamphoacetate, disodium lauroamphodiacetate, sodium cocoamphoacetate, disodium cocoamphodiacetate, disodium soyamphodiacetate, disodium wheatamphodiacetate, and sodium cocoabutter amphoacetate.

In a second embodiment, preferred compounds of formula (I) are alkylamphosulfonates, that may be chosen in the group consisting of: sodium lauroamphosulfonate and sodium cocoampho hydroxypropyl sulfonate.

In a third embodiment of the present invention, preferred compounds of formula (I) are alkyl betaines and alkylamidopropyl betaines, such as cocoamidopropyl betaine (CAPB), lauramidopropyl betaine, coco-betaine, lauryl betaine or cetyl betaine.

In a fourth embodiment of the present invention, preferred compounds of formula (I) are alkyl or alkylamidopropyl hydroxysultaines, such as cocamidopropyl hydroxysultaine, lauramidopropyl hydroxysultaine, laurel hydroxysultaine

It has to be noticed that the compound of formula (I) may be an imidazoline derived compound.

### Component (3)

Cationic polymer derivative of polysaccharide of the present invention are preferably cationic guars. Cationic guars may include cationic guars that may be obtained by the use of different possible cationic etherifying agents, such as for example the family of quaternary ammonium salts.

In the case of cationic guars, the cationic group may be then a quaternary ammonium group bearing three radicals, which may be identical or different, chosen from hydrogen, an alkyl radical containing 1 to 22 carbon atoms, more particularly 1 to 14 and advantageously 1 to 3 carbon atoms. The counterion is generally a halogen, such as chlorine.

Quaternary ammonium salts may be for example: 3-chloro-2-hydroxypropyl trimethyl ammonium chloride (CHPTMAC), 2,3-epoxypropyl trimethyl ammonium chloride (EPTAC), and diallyldimethyl ammonium chloride (DMDAAC).

A typical cationic functional group in these cationic guar derivatives is trimethylamino(2-hydroxyl)propyl, with a counter ion. Various counter ions can be utilized, including but not limited to halides, such as chloride, fluoride, bromide, and iodide, sulfate, methylsulfate, and mixtures thereof.

Cationic guars of the present invention may be chosen in the group consisting of:
cationic hydroxyalkyl guars, such as cationic hydroxyethyl guar (HE guar), cationic hydroxypropyl guar (HP guar), cationic hydroxybutyl guar (HB guar), and
cationic carboxylalkyl guars including cationic carboxymethyl guar (CM guar), cationic alkylcarboxy guars such as cationic carboxylpropyl guar (CP guar) and cationic carboxybutyl guar (CB guar), carboxymethylhydroxypropyl guar (CMHP guar).

More preferably, cationic guars of the invention are guars hydroxypropyltrimonium chloride.

The Degree of Substitution (DS) of cationic guars, that is the average number of hydroxyl groups that have been substituted by a cationic group per monosaccharide unit, may be comprised between 0.005 and 3, preferably between 0.01 and 2. DS may notably represent the number of the carboxymethyl groups per monosaccharide unit. DS may notably be determined by titration.

The cationic guar may have an average Molecular Weight (Mw) of between about 100,000 daltons and 3,500,000 daltons, preferably between about 500,000 daltons and 3,500,000 daltons.

Preferably the soap based composition comprises at least:

(1) 5 to 35% by weight of carboxylic acids having 8 to 24 carbon atoms, and salts thereof;
(2) 0.5 to 15% by weight of a compound of formula (I), preferably an alkylamphocarboxylate; and
(3) 0.02 to 2% by weight, of a cationic polymer derivative of polysaccharide, preferably cationic guar;
1 to 15% by weight, of one or more anionic surfactants
0.1 to 10% by weight, of one ore more nonionic surfactants; and
0.1 to 10% by weight, an organic, inorganic or polymeric stabilizer. and each components of the composition are expressed in percent by weight in relation with the total weight of the composition.

### Other Compounds

The foaming composition of the present invention can also comprise other components such as surfactants, organic or inorganic thickeners (such as hydroxyethyl cellulose, HEC), opacifying or pearlescent agents (for example ethyleneglycol distearate, EGDS), water-insoluble skin benefit agents, exfoliating particles, preservatives, polymers with skin, hair or foam benefits, antimicrobials, bactericides, antioxydants (such as butylated hydroxytoluene, BHT), humectants and emmolients, refatting agents, solvents like polyhydric alcohols, fragrances, colouring agents and sequestering agents (for example sodium salt of the ethylenediaminetetraacetic acid, 4NaEDTA).

The surfactants in the composition may be selected from any known anionic, cationic, nonionic and amphoteric/zwiterionic surfactants suitable for applications to the human body.

Anionic surfactants may be alkyl sulfates and alkyl ether sulfates of formula: R—(OCH₂CH₂)ₙ—SO₄M; wherein R is an alkyl or alkenyl group having 8 to 22 carbons, preferably 12 to 18 carbons and M is a cation such as sodium, potassium or ammonium; n ranges from 0 to 10.

The anionic surfactants may also be aliphatic sulphonates, such as primary alkane or alkene (e.g. C₈-C₂₂) sulphonate or disulphonate, alkylglyceryl ether sulphonate or aromatic sulphonate.

Other possible anionic surfactants include:
sulfosuccinates having the formula: R—(CONH)n-(OCH2CH2)m-O₂CCH₂CH(SO₃M)-CO₂M, wherein R is alkyl or alkenyl ranging from C₇ to C₂₁ and M is a solubilizing cation; n could be 0 or 1 and m has an average value between 0 and 5.
Taurates with the formula R—CONR₁CH₂CH₂SO₃M, wherein R is C₇ to C₂₁ alkyl or alkenyl; R₁ is C1 to C4 alkyl and M is a solubilizing cation.
Isethionates which are generally identified by the formula: R—COOCH₂CH₂SO₃M; wherein R is alkyl or alkenyl ranging from C₇ to C₂₁ and M is a solubilizing cation;

Another possible class of anionic surfactants is the phosphate esters as follows: R—(OCH₂CH₂)n-PO₄M₂ or (R—(OCH2CH2)n)m-PO₄M wherein R is alkyl or alkenyl ranging from C₈ to C₂₂ and M is a solubilizing cation; m could be 1 or 2 and n has an average value between 0 and 5.

Also included are the carboxylates having the formula R—(CH₂CH₂O)n-OCH₂CO₂M; wherein R is alkyl or alkenyl ranging from C₈ to C₂₂ and M is a solubilizing cation; n has an average value between 0 and 15.

Preferred non-ionic surfactants are:
The monoethanol, diethanol, monoisopropanol and methylmonoethanol amides of fatty acids having an acyl moiety of from 8 to about 18 carbon atoms, such as coconut monoethanolamide (CMEA).
The condensation products of alkyl phenol or aliphatic primary or secondary, linear or branched alcohols with ethylene oxide. Typically they have a carbon chain ranging from 8 to 22 carbon atom and 5 to 30 moles of ethylene oxide per mole of alcohol.
Long chain tertiary amine oxides corresponding to the following general formula R¹R²R³NO wherein R¹ is an alkyl radical of from about 8 to about 24 carbon atoms, R² and R³ are each methyl, ethyl or hydroxyethyl radicals.
Alkylpolysaccarides, particularly alkylpolyglucosides composed of a polyglycosyl moiety of 1 to 10 units linked to the normal-chain or branched-chain alkyl, alkenyl or acyl moiety of from 8 to 18 carbon atoms.

Preferred amphoteric or zwiterionic surfactants are:
betaines with formula:

R¹R²R³N⁺R⁴C(O)O⁻
amidoalkyl betaines with formula:

R¹C(O)—N(H)R²N⁺(R³R⁴)R⁵C(O)O⁻
or sulphobetaines with formulas:

R¹R²R³N⁺R⁴SO₃⁻

and

R¹C(O)—N(H)R²N⁺(R³R⁴)R⁵SO₃⁻

where R¹ represents an alkyl or alkenyl radical containing 7 to 21 carbon atoms, R², R³, R⁴ and R⁵ are alkyl or alkylene radicals of from 1 to 4 carbon atoms.
amphoacetates with formula:

R¹C(O)—N(H)R²N⁺(R⁶R⁷)R⁵C(O)O⁻

where R¹ represents an alkyl or alkenyl radical containing 7 to 21 carbon atoms, R² and R⁵ are alkyl or alkylene radicals of from 1 to 4 carbon atoms, R⁶ is CH₂CH₂OH, R⁷ is H or R⁵C(O)O⁻
amphosulphonates with formula:

R¹C(O)—N(H)R²N⁺(H)(CH₂CH₂OH)(CH₂CH(OH)CH₂SO₃⁻)

where R¹ represents an alkyl or alkenyl radical containing 7 to 21 carbon atoms and R² is alkyl or alkylene radical of from 1 to 4 carbon atoms,

### Preparation of the Composition

Components of the composition may be blended in several possible ways, notably in or several successive steps. As example, it's possible to mix components (1), (2) and (3) all together, notably in water. It's also possible to first blend component (1) and component (2) and then further blend the resulting mixture with component (3). Also it is possible to add the component (3) into component (2) and than to mix with component (1). The component (3) could be predispersed in polyhydric alcohols like glycerine or propylene glycol for easier blending. Other components may be added during or after each of these steps.

The examples provided here further describe and demonstrate embodiments of the present invention. The examples are given solely for the purpose of illustration and are not to be construed as limitation of the present invention

### EXPERIMENTAL PART

Used guar hydroxypropyl trimonium chloride is having a molecular weight of approximately 2000000, degree of substitution between 0.1 and 0.13 and charge density about 0.6-0.7 meq/grams.

### Procedure

All examples were prepared according to the typical procedure described below:

1) Mixture of fatty acids and BHT was heated to 75° C. until becoming fluid (mixture-1);
2) Mixture of potassium hydroxide, glycerine and water was heated to 75° C. (mixture-2);
3) Mixture 2 was added to mixture-1 with agitation (mixture-3);
4) SLES and 4NaEDTA, pre-dissolved in water, and CMEA (if present) were added to mixture-3 (mixture-4).
5) EGDS was added into mixture-4 and after it was dissolved the mixture was cooled to 50° C.;
6) Amphoteric surfactant dissolved in water was added into mixture-4 (mixture-5);
7) A dispersion of cationic guar polymer, together with the thickening polymer, e.g. HEC in propylene glycol was added into mixture-5 and the temperature was decreased to 30° C. while stirring (mixture-6);
8) Preservative was then added.

### Protocols

### Foam Volume and Foam Drainage Test

A solution containing 2% by wt. of the composition in water containing 50 ppm CaCl₂ was prepared and stirred for 5 minutes. Than a 200 g of this solution were transferred in the jar of a kitchen blender NATIONAL model MX-795N. The solution was stirred for 15 seconds and transferred carefully in a 1000 ml measuring cylinder. The foam volume was recorded and the foam drainage, i.e. the volume of the liquid below the foam was tracked for 15 minutes. The foam drainage value mentioned in this document is the volume of the liquid 5 minutes after the transfer into the measuring cylinder. At least two experiments were done and the average value was taken. When the volume is bigger and the drainage value is smaller than the foam is considered better.

### Foam Quality Test

A solution of the composition in water containing 50 ppm CaCl₂ was prepared and stirred for 5 minutes. Unless otherwise stated the concentration of the solution was 3% by wt. After that the solution was foamed in the abovementioned kitchen blender for 1 minute and the produced foam was transferred in a funnel placed on a laboratory sieve No 18 with 1 mm mesh size, unless otherwise stated. The funnel was plastic with 150 mm diameter, 25 mm opening and 125 mm height. It has a steel wire at 67 mm height which serves as a mark. When the foam from the blender is poured into the funnel it goes through the sieve into a stainless steel container. The time which is needed for the foam surface to reach the metal wire is recorded. Foam with longer residence time is considered better. All experiments were made at least two times and the results were averaged. Sometimes the different compositions required different concentrations and/or mesh size for this test in order to be able to better distinguish between them and this is indicated in the results provided. This test provide information about the foam viscosity and foam elasticity, as well for the foam-surface friction, which are among the main factors affecting the consumer perception about the foam quality. Results are expressed in Tables 1 and 2.

TABLE 1

| | Formulations | | | |
|---|---|---|---|---|
| | C1 | C2 | 1 | 2 |
| Components | | | | |
| Lauric Acid | 7 | 7 | 7 | 7 |
| Myristic Acid | 2 | 2 | 2 | 2 |
| Palmitic Acid | 1 | 1 | 1 | 1 |
| Potassium Hydroxide | 2.74 | 2.74 | 2.74 | 2.74 |
| Sodium laurylether sulfate (SLES) | 2 | 2 | 2 | 2 |
| Sodium lauroamphoacetate | | 2 | | 2 |
| (SLAA) | | | | |
| Cocamidopropyl betaine (CAPB) | 2 | | 2 | |
| Ethylene glycol distearate (EGDS) | 1 | 1 | 1 | 1 |
| Propylene Glycol (PG) | 1 | 1 | 1 | 1 |
| Hydroxyethyl cellulose (HEC) | 0.75 | 0.75 | 0.6 | 0.6 |
| Guar hydroxypropyl trimonium | | | 0.15 | 0.15 |
| chloride (C14S) | | | | |
| Tetrasodium edentate (EDTA) | 0.1 | 0.1 | 0.1 | 0.1 |
| Preservative | 0.22 | 0.22 | 0.22 | 0.22 |
| Dibutylhydroxytoluene (BHT) | 0.05 | 0.05 | 0.05 | 0.05 |
| Water | to 100 | to 100 | to 100 | to 100 |
| Properties | | | | |
| Foam volume | 620 | 723 | 675 | 800 |
| Foam drainage | 108.5 | 82.7 | 82.5 | 50 |
| Foam quality | 15.3 | 174.7 | 343.3 | >900 |
| (3.3%; 1.7 mm mesh) | | | | |

TABLE 2

| | Formulations | | | | |
|---|---|---|---|---|---|
| | C3 | C4 | C5 | 3 | 4 |
| Components | | | | | |
| Lauric Acid | 5.4 | 4.5 | 4.5 | 4.5 | 4.5 |
| Myristic Acid | 6 | 5 | 5 | 5 | 5 |
| Palmitic Acid | 0.6 | 0.5 | 0.5 | 0.5 | 0.5 |
| Potassium Hydroxide | 3.02 | 2.72 | 2.72 | 2.72 | 2.72 |
| Sodium laurylether | 1 | 1 | 1 | 1 | 1 |
| sulfate | | | | | |
| Sodium | | 2 | | | 2 |
| lauroamphoacetate | | | | | |
| Cocamidopropyl betaine | | | 2 | 2 | |
| Cocoamide | 1 | 1 | 1 | 1 | 1 |
| monoethanolamide | | | | | |
| Ethylene glycol | 1 | 1 | 1 | 1 | 1 |
| distearate | | | | | |
| Glycerine | 4 | 4 | 4 | 4 | 4 |
| Propylene Glycol | 1 | 1 | 1 | 1 | 1 |
| Hydroxypropyl guar | 0.75 | 0.75 | 0.75 | 0.6 | 0.6 |
| Guar hydroxypropyl | | | | 0.15 | 0.15 |
| trimonium chloride | | | | | |
| Tetrasodium edentate | 0.1 | 0.1 | 0.1 | 0.1 | 0.1 |
| Preservative | 0.22 | 0.22 | 0.22 | 0.22 | 0.22 |
| BHT | 0.05 | 0.05 | 0.05 | 0.05 | 0.05 |
| Water | to 100 | to 100 | to 100 | to 100 | to 100 |
| Properties | | | | | |
| Foam volume | 590 | 690 | 632 | 645 | 743 |
| Foam drainage | 117.5 | 87.7 | 103 | 82.5 | 63.5 |
| Foam quality | 24 | 76.3 | 13.7 | 340.3 | >900 |

It appears then that the formulation of the present invention comprising components (1), (2) and (3) permits to obtain a voluminous foam with excellent foam quality.

1. Use of a combination of a compound of formula (I) and a cationic polymer derivative of polysaccharide to increase the foam volume and foam quality of a soap containing aqueous composition; said composition comprising at least:
(1) 0.5 to 85% by weight of carboxylic acids having 8 to 24 carbon atoms, and salts thereof;
(2) 0.5 to 15% by weight of an amphoteric surfactant of formula (I) as follows:
R¹—(CONH)ₐ—(CH₂)_{b}—N⁺(R²)(R³)(R⁴)Z⁺ (I)
wherein:
a is 0 or 1;
b is comprised between 1 and 3;
R¹ is an alkyl or alkenyl hydrocarbon chain comprising from 7 to 21 carbon atoms;
R² is an alkyl group having 1 to 3 carbon atoms or —(CH₂)_{c}OH with c is comprised between 1 and 3;
R³ is H, an alkyl group having 1 to 3 carbon atoms, or —(CH₂)_{d}COO⁻ with d is comprised between 1 and 3;
R⁴ is —(CH₂)ₑCOO⁻ with e is comprised between 1 and 3; or —(CH₂)_{f}CH(OH)—CH₂—SO₃⁻ with f is comprised between 1 and 3; and
Z is a monovalent cation.
(3) 0.01 to 5% by weight of a cationic polymer derivative of polysaccharides;
and each components of the composition are expressed in percent by weight in relation with the total weight of the composition.

2. The use according to claim 1, wherein said components (1) are the alkali metal or alkanol ammonium salts of aliphatic alkane- or alkene monocarboxilic acids

3. The use according to claim 1, wherein the monovalent cation Z is an alkaline metal such as sodium and potassium, or mono- di- or tri-ethanolammonium.

4. The use according to claim 1, wherein said compound of formula (I) is selected from the group consisting of:
alkylamphocarboxylates, alkylamphosulfonates, alkyl or alkylamidopropyl hydroxysultaine and alkyl or alkylamidopropyl betaines.

5. The use according to claim 1, wherein said compound of formula (I) is selected from the group consisting of: sodium lauroamphoacetate, disodium lauroamphodiacetate, sodium cocoamphoacetate, disodium cocoamphodiacetate, disodium soyamphodiacetate, disodium wheatamphodiacetate, and sodium cocoabutter amphoacetate.

6. The use according to claim 1, wherein said compound of formula (I) is selected from the group consisting of: sodium lauroamphosulfonate and sodium cocoampho hydroxypropyl sulfonate.

7. The use according to claim 1, wherein said compound of formula (I) is selected from the group consisting of:
cocoamidopropyl betaine, lauramidopropyl betaine, coco-betaine, lauryl betaine and cetyl betaine.

8. The use according to claim 1, wherein said compound of formula (I) is selected from the group consisting of cocamidopropyl hydroxysultaine, lauramidopropyl hydroxysultaine, and laurel hydroxysultaine

9. The use according to claim 1, wherein said components (3) are cationic guars.

10. The use according to claim 9, wherein said cationic guars are selected from the group consisting of:
cationic hydroxyalkyl guars, such as cationic hydroxyethyl guar, cationic hydroxypropyl guar, cationic hydroxybutyl guar, and
cationic carboxylalkyl guars including cationic carboxymethyl guar, cationic alkylcarboxy guars such as cationic carboxylpropyl guar and cationic carboxybutyl guar, carboxymethylhydroxypropyl guar.

11. A soap containing composition comprising at least:
(1) 5 to 35% by weight of carboxylic acids having 8 to 24 carbon atoms, and salts thereof;
(2) 0.5 to 15% by weight of a compound of formula (I)
R¹—(CONH)ₐ—(CH₂)_{b}—N⁺(R²)(R³)(R⁴)Z⁺ (I)
wherein:
a is 0 or 1;
b is comprised between 1 and 3;
R¹ is an alkyl or alkenyl hydrocarbon chain comprising from 7 to 21 carbon atoms;
R² is an alkyl group having 1 to 3 carbon atoms or —(CH₂)_{c}OH with c is comprised between 1 and 3;
R³ is H, an alkyl group having 1 to 3 carbon atoms, or —(CH₂)_{d}COO⁻ with d is comprised between 1 and 3;
R⁴ is —(CH₂)ₑCOO⁻ with e is comprised between 1 and 3; or —(CH₂)_{f}CH(OH)—CH₂—SO₃⁻ with f is comprised between 1 and 3; and
Z is a monovalent cation.
(3) 0.02 to 2% by weight, of a cationic polymer derivative of polysaccharide; and
1 to 15% by weight, of one or more anionic surfactants
0.1 to 10% by weight, of one ore more nonionic surfactants; and
0.1 to 10% by weight, an organic, inorganic or polymeric stabilizer and
and each components of the composition are expressed in percent by weight in relation with the total weight of the composition.

12. A method to produce foaming product by using a composition comprising at least components (1), (2) and (3) identified in claim 1.
